(19) United States
(12) Patent Application Publication
(10) Pub. No.: US 2026/0191573 A1
(43) Pub. Date: Jul. 9, 2026

(54) DYNAMIC COMPRESSION DEVICES AND PROCESSES FOR MAKING AND USING SAME

(71) Applicant: MedShape, Inc., Atlanta, GA (US)

(72) Inventors: Adam DeBosier, Atlanta, GA (US); Daniel Lane Jobe, Kennesaw, GA (US); Cody Benjamin Kraner, Marietta, GA (US); Ryan Walter O'Flaherty, Atlanta, GA (US); David Lee Safranski, Atlanta, GA (US); Tyler Joseph Touchet, Woodland Park, CO (US); Jeremy Webster Blair, Atlanta, GA (US); Ian Perry McClellan, Lawrenceville, GA (US); Donald Kenneth Griffin, Marietta, GA (US)

(21) Appl. No.: 19/557,092

(22) Filed: Mar. 4, 2026

Related U.S. Application Data
(63) Continuation-in-part of application No. 19/468,652, filed on Jan. 1, 0001.
Application No. 19/468,652 is a continuation of application No. 18/355,373, filed on Jul. 19, 2023, now Pat. No. 12,569,285.
Application No. 18/355,373 is a continuation of application No. 17/711,865, filed on Apr. 1, 2022, now Pat. No. 11,744,625.
Application No. 17/711,865 is a division of application No. 17/184,104, filed on Feb. 24, 2021, now Pat. No. 11,291,488.
(60) Provisional application No. 63/766,582, filed on Mar. 4, 2025.

(51) Int. Cl. A61B 17/86; A61B 17/56; A61B 17/68; A61B 17/72

(52) U.S. Cl. CPC A61B 17/864; A61B 17/863; A61B 2017/564; A61B 2017/681; A61B 17/7225; A61B 17/8625; A61B 2017/8655; A61B 17/8685

(57) ABSTRACT

A compression device may include, but is not limited to, a threaded body, a sliding element, a setscrew, and a compression element connecting the threaded body and the sliding element. According to one embodiment, upon implantation, the threaded body contacts a first skeletal element and the sliding element contacts a second skeletal element. In at least one embodiment, upon being engaged, the compression element applies sustained tension to the sliding element and opposing tension to the threaded body, thereby compressing the first skeletal element and the second skeletal element along a plane of contact to promote healing.

### CROSS-REFERENCE TO RELATED APPLICATIONS

This application:
is a continuation-in-part of U.S. patent application Ser. No. 19/468,652, filed Feb. 3, 2026, entitled “DYNAMIC COMPRESSION DEVICES AND PROCESSES FOR MAKING AND USING SAME,” which is a continuation of U.S. patent application Ser. No. 18/355,373, filed Jul. 19, 2023, entitled “DYNAMIC COMPRESSION DEVICES AND PROCESSES FOR MAKING AND USING SAME,” which is a continuation of U.S. patent application Ser. No. 17/711,865, filed Apr. 1, 2022, now U.S. Pat. No. 11,744,625, entitled “DYNAMIC COMPRESSION DEVICES AND PROCESSES FOR MAKING AND USING SAME,” which is a divisional of U.S. patent application Ser. No. 17/184,104, filed Feb. 24, 2021, now U.S. Pat. No. 11,291,488, entitled “DYNAMIC COMPRESSION DEVICES AND PROCESSES FOR MAKING AND USING SAME”; and
claims priority to U.S. provisional patent application No. 63/766,582 filed March 4, 2025, entitled “DYNAMIC COMPRESSION DEVICES AND PROCESSES FOR MAKING AND USING SAME.”

The contents of the above applications are incorporated herein by reference in their entireties.

### BACKGROUND

Orthopedic conditions such as fractures and joint fusion procedures may be treated, in part, using sustained dynamic at the fracture site or a joint. Joint fusion typically involves compressing two or more skeletal elements together to promote ossification, resettlement, and fusion processes. Previous joint fusion approaches include a threaded compression device that is inserted into and compresses together two or more skeletal elements. In such approaches, compressive forces are typically generated by threaded elements extending along at least a portion of the compression device.

However, these previous approaches typically suffer compression performance issues, such as insufficient or discontinuous compression. As one example, a surgeon inserts a threaded compression device into two or more skeletal elements, thereby generating a compressive force at a treatment site therebetween. In response to the compressive force, the two or more skeletal elements resettle and undergo resorption, thereby substantially reducing or fully dissipating the static compressive force. The inability of the threaded compression device to adapt to changes at the insertion site and provide a dynamic compressive force may result in insufficient healing at the treatment site.

Therefore, there is a long-felt but unresolved need for a sustained dynamic compression device that allows for generation of sustained dynamic compressive forces between skeletal elements, including but not limited to fracture fixation and joint fusion applications.

### BRIEF SUMMARY OF THE DISCLOSURE

Briefly described, and according to one embodiment, aspects of the present disclosure generally relate to devices and assemblies for providing sustained dynamic compression of skeletal structures as well as processes for making and using the same.

In one or more embodiments, a compression device provides sustained compression to a target site by maintaining a predetermined level of tension between a first device portion that is configured to contact a first skeletal element and a second device portion configured to contact a second skeletal element.

In one or more embodiments, a compression device includes, but is not limited to, a threaded body, a setscrew, a sliding element, and a compression element connecting the threaded body and the sliding element.

According to one embodiment, the compression element includes a superelastic material, such as nitinol, and is configured to generate sustained dynamic compressive forces between skeletal elements, including but not limited to bone fragments at a fracture site, adjacent bones at a joint targeted for fusion, and/or a tendon-to-bone interface.

In at least one embodiment, the threaded body is cannulated such that a portion of the compression element is sheathed by the threaded body, and the threaded body and sliding element include internal fittings for connecting to corresponding fittings on each end of the compression element.

In one or more embodiments, the compression element, threaded body, and sliding element are cannulated such that the compression device is insertable along a guidewire to a target site.

In some embodiments, the compression element is in a deformed (e.g., stretched) state prior to insertion, and the compression device is configured such that, after implantation, compression is applied via the compression element returning toward a relaxed state from the deformed state, thereby applying sustained opposing forces through the sliding element and the threaded body to compress the skeletal elements.

In one or more embodiments, the compression device is configured for controlled pre-tensioning of the compression element using an insertion tool and a connection bolt. In one example, stretching includes securing a position of the sliding element while applying a force to the threaded body via one or more pins of an insertion tool, thereby translating the threaded body away from the stationary sliding element and stretching the compression element. In this example, the stretched position may be secured by inserting a connection bolt through a hollow interior of the insertion tool and attaching the connection bolt to the sliding element to preserve the stretched/deformed state of the compression element prior to implantation.

In some embodiments, stretching of the compression element occurs during insertion of the compression device to a target site (e.g., by rotating the compression device into the target site until a predetermined level of stretch is achieved), and the compression element dynamically responds to movement and structural changes at the target site (including resettling and resorption) to maintain substantially continuous compression between skeletal elements.

In various embodiments, components of the compression device and associated tools are formed of biocompatible materials, including, by way of example, titanium and/or titanium alloys for the threaded body, sliding element, insertion tool, and/or related components, and nitinol for the compression element.

According to a first aspect, a compression device assembly includes an elongate threaded body defining a hollow interior, a sliding element defining a hollow interior and configured to contact a second skeletal element, a setscrew, and a nitinol compression element operatively connected to the threaded body and the sliding element, wherein the nitinol compression element is in a deformed state prior to insertion and the assembly is configured to apply compression via the nitinol compression element returning toward a relaxed state.

According to a second aspect, the compression device assembly of the first aspect or any other aspect, includes a nitinol compression element having a second end comprising a slotted end configured to receive an insert.

According to a third aspect, the compression device assembly of any aspects herein includes a threaded body having a tapered shaft, wherein the shaft tapers from a proximal region toward a distal region.

According to a fourth aspect, the compression device assembly of any aspects herein is cannulated for insertion over a guidewire, including via cannulation extending through the nitinol compression element, threaded body, and sliding element.

According to a fifth aspect, the compression device assembly of any aspects herein includes an insertion tool having one or more pins configured to be received by one or more pin openings and/or pin slots of the sliding element, wherein a pin length is selected so that a maximum stretch length of the nitinol compression element does not exceed a failure stretch length.

According to a sixth aspect, the compression device assembly of any aspects herein includes an insertion tool defining a hollow interior configured to receive a connection bolt through the insertion tool, wherein the connection bolt is configured to attach to the sliding element to preserve the deformed state of the nitinol compression element prior to implantation.

According to a seventh aspect, the present disclosure includes methods of assembling, stretching, securing, inserting, and engaging the compression device assembly such that, after implantation, disengaging and/or withdrawing the insertion tool and allowing the nitinol compression element to return toward a relaxed state applies compression between first and second skeletal elements.

According to an eighth aspect, a kit includes the compression device assembly, the insertion tool, and a connection bolt, wherein the connection bolt, nitinol compression element, and setscrew are configured to interface with corresponding threads of the sliding element, and the nitinol element passes through the threaded body.

### BRIEF DESCRIPTION OF THE DRAWINGS

FIG. 1 is a perspective view of an exemplary compression assembly, according to one embodiment of the present disclosure.

FIG. 2 is a cross-sectional view of an exemplary compression assembly, according to one embodiment of the present disclosure.

FIG. 3 is a perspective view of an exemplary compression assembly, according to one embodiment of the present disclosure.

FIG. 4 is a cross-sectional view of a connection bolt, according to one embodiment of the present disclosure.

FIG. 5 is a cross-sectional view of an example insertion tool, according to one embodiment of the present disclosure.

FIG. 6 is a cross-sectional view of an example sliding element, according to one embodiment of the present disclosure.

FIG. 7 is a cross-sectional view of an example compression element, according to one embodiment of the present disclosure.

FIG. 8 is a cross-sectional view of an example setscrew, according to one embodiment of the present disclosure.

FIG. 9 is a cross-sectional view of an example threaded body, according to one embodiment of the present disclosure.

FIG. 10 is a side view of components of an exemplary compression device assembly, according to one embodiment of the present disclosure.

FIG. 11 is an alternate side view of components of an exemplary compression device assembly, according to one embodiment of the present disclosure.

FIG. 12 is a perspective view of an exemplary compression assembly, according to one embodiment of the present disclosure.

FIG. 13 is a perspective view of an exemplary compression assembly, according to one embodiment of the present disclosure.

FIG. 14 is a cross-sectional view of an exemplary compression assembly, according to one embodiment of the present disclosure.

FIG. 15 is a perspective view of an exemplary compression assembly, according to one embodiment of the present disclosure.

FIG. 16 is a cross-sectional view of a connection bolt, according to one embodiment of the present disclosure.

FIG. 17 is a cross-sectional view of an example insertion tool, according to one embodiment of the present disclosure.

FIG. 18 is a cross-sectional view of an example sliding element, according to one embodiment of the present disclosure.

FIG. 19 is a cross-sectional view of an example setscrew, according to one embodiment of the present disclosure.

FIG. 20 is a cross-sectional view of an example compression element, according to one embodiment of the present disclosure.

FIG. 21 is a cross-sectional view of an example threaded body, according to one embodiment of the present disclosure.

FIG. 22 is a side view of components of an exemplary compression device assembly, according to one embodiment of the present disclosure.

FIG. 23 is a cross-sectional view of components of an exemplary compression device assembly, according to one embodiment of the present disclosure.

FIG. 24 is a perspective view of components of an exemplary compression device assembly, wherein the threaded body is shown as transparent, according to one embodiment of the present disclosure.

FIG. 25 is a perspective view of components of an exemplary compression device assembly, according to one embodiment of the present disclosure.

FIG. 26 shows an exemplary process, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

In various embodiments, any of the components, assemblies, methods, and configurations described herein may be used in combination with, or interchanged with, corresponding components, assemblies, methods, and configurations disclosed in U.S. Pat. No. 11,291,488, the entire contents of which are incorporated herein by reference.

### Overview

Aspects of the present disclosure generally relate to devices and assemblies for providing precise and accurate compression of skeletal structures in applications including, but not limited to, fracture fixation and joint fusion, as well as processes for making and using the same. In one or more embodiments, a compression device includes, but is not limited to, a threaded body, a sliding element, and one or more compression elements.

In one or more embodiments, a compression device includes, but is not limited to, a threaded body, a sliding element, and one or more compression elements. In at least one embodiment, the threaded body is cannulated such that a portion (or, in some embodiments, a substantial length) of the compression element is sheathed by the threaded body. In one or more embodiments, the threaded body and the sliding element include internal fittings for connecting to corresponding fittings on each end of a compression element. In one example, the threaded body and the sliding element each include threads sized to receive corresponding threads of the compression element.

In at least one embodiment, the compression element provides for generation of sustained dynamic compressive forces between two or more skeletal elements, including, but not limited to, bone fragments at a fracture site, adjacent bones at a joint, a tendon and its attachment or insertion to a bone (e.g., a tendon-to-bone interface), or other skeletal elements at which sustained dynamic compression is desired to promote healing, ossification, and/or fusion (e.g., along a plane of fracture, joint interface, or tendon-to-bone interface through which the compression device is inserted). According to one embodiment, the compression element is cannulated according to a predetermined diameter. In some embodiments, the compression element is not cannulated. In at least one embodiment, the cannulation of the compression element, threaded body, and sliding element permit the compression device to be inserted along a guidewire (e.g., translated through the cannulated portions) into a target site, such as a fracture site or a joint targeted for fusion. In at least one embodiment, the cannulation of the compression element allows for pre-configuration of varying compression levels without affecting an overall footprint of the compression element, thereby enabling the compression device to be tailored to a range of clinical applications, including fracture fixation, bone fusion, and joint fusion.

In various embodiments, the compression element includes a superelastic material, such as nitinol. In one or more embodiments, the threaded body, sliding element, insertion tool, connection rod, and/or connecting pins include one or more materials including, but not limited to, titanium, titanium alloys, and other materials.

By way of example and not limitation, the elements presented in connection with compression device assembly 3004 (e.g., threaded body 351, sliding element 335, compression element 340, insertion tool 310, connection bolt 303, and setscrew 345) are interchangeable with the corresponding elements of compression device assembly 4004 (e.g., threaded body 451, sliding element 435, compression element 440, insertion tool 410, connection bolt 403, and setscrew 445), and vice versa, as would be appreciated by one of ordinary skill in the art.

### EXEMPLARY EMBOIMENTS

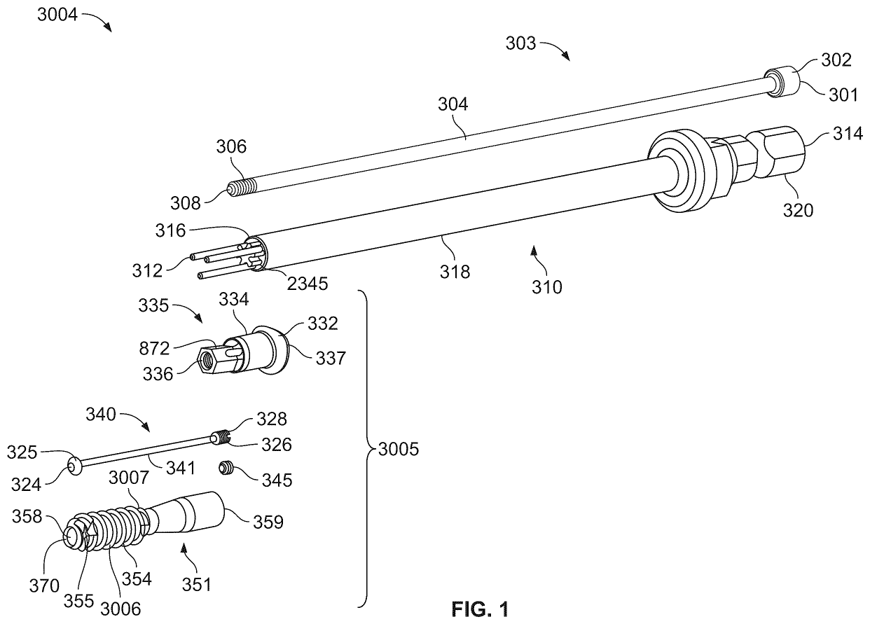

Referring now to the figures, for the purposes of example and explanation of the fundamental processes and components of the disclosed systems and methods, reference is made to FIG. 1, which shows a perspective view of an exemplary compression assembly 3004 according to various embodiments of the present disclosure. As will be understood and appreciated, the exemplary compression assembly 3004 shown in FIG. 1 represents merely one approach or embodiment of the present system, and other aspects are used according to various embodiments of the present system.

FIG. 1, which shows a perspective view of an exemplary compression assembly 3004 according to various embodiments of the present disclosure. As will be understood and appreciated, the exemplary compression assembly 3004 shown in FIG. 1 represents merely one approach or embodiment of the present system, and other aspects are used according to various embodiments of the present system.

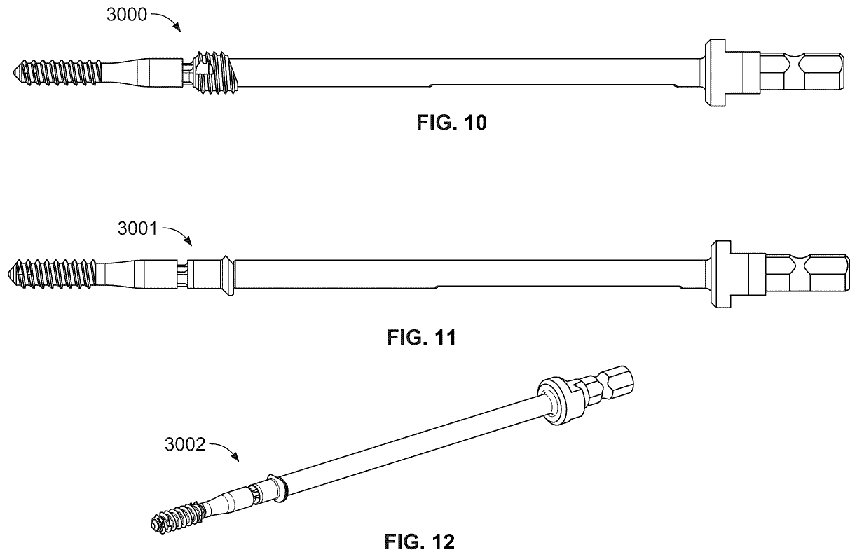

In various embodiments, the assembly 3004 includes a compression device 3005, an insertion tool 310, and a connection bolt 303. In some embodiments, the compression device 3005, the insertion tool 310, and the connection bolt 303 are provided separately (e.g., unattached or unassembled), such as, for example, in a kit. According to one embodiment, the compression device 3005 and insertion tool 310 are provided as shown in FIG. 1 or FIG. 10, and the compression device 3005 is provided stretched according to predetermined parameters. In alternate embodiments, the compression device 3005 and insertion tool 310 are provided as the assembly 3004, but the compression device 3005 is not stretched (e.g., the stretching being performed by a user, such as a technician or surgeon). In some embodiments, the compression device 3005 is insertable without the insertion tool 310.

In various embodiments, the connection bolt 303 comprises including a second end 301, a head 302, a shaft 304, a connection mechanism 306, and a first end 308. In some embodiments, the connection bolt 303 shaft 304 comprises a hollow interior and/or cannulated region. In other embodiments, the connection bolt 303 shaft 304 is not cannulated and/or comprises a solid interior. According to one embodiment, the connection bolt 303 comprises a substantially cylindrical shape. In other embodiments, the connection bolt 303 comprises one or more shapes including, but not limited to circles, semi-circles, hexagons, and other polygons. In some embodiments, the connection bolt 303 is cannulated and/or comprises a hollow interior. In some embodiments, the connection bolt 303 is not cannulated and/or does not comprise a hollow interior. In some embodiments, the connection mechanism 306 comprises threads. In some embodiments, the connection mechanism 306 is configured to interface with the sliding element 335. For instance, in some embodiments, the connection mechanism 306 comprises screw threads that can engage with the internal threads 339 of the sliding element.

According to one embodiment, the connection bolt 303 is provided in a kit (e.g., including the components of the compression assembly 3004) and a user inserts and rotates the connection bolt 303 to achieve a desired tensioning of the compression device 3005. In at least one embodiment, the connection bolt 303 is inserted into the compression assembly 3004 during an assembly process. In one or more embodiments, the tensioning of the compression element 340 is performed according to predetermined implantation parameters, for example, a desired compression force to be applied to skeletal elements of a patient.

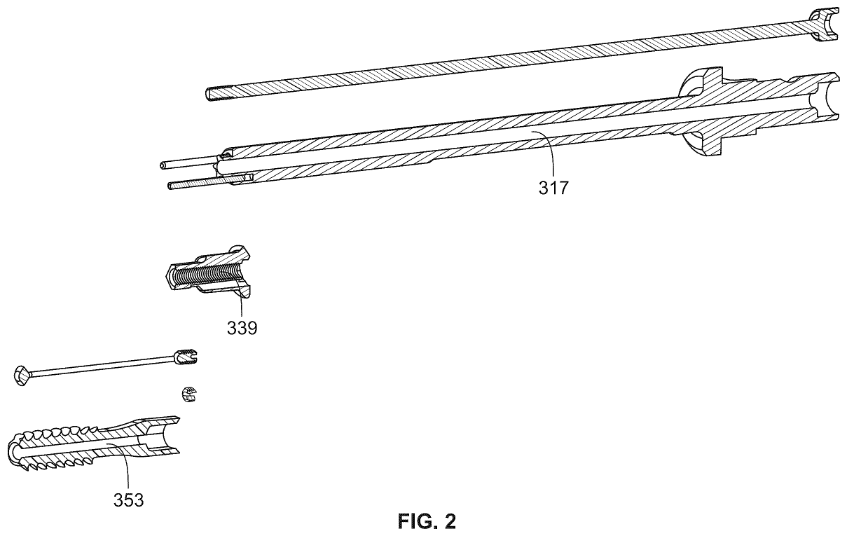

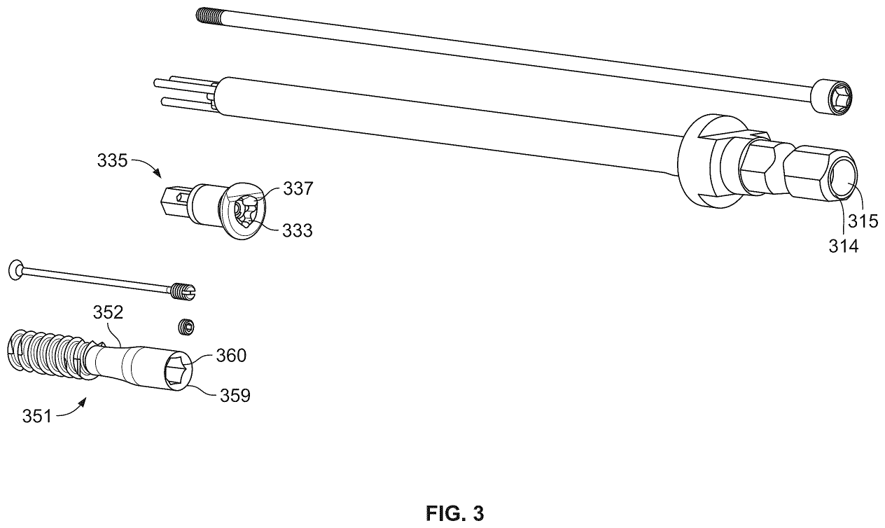

In one or more embodiments, the insertion tool 310 includes a first end 316, a second end 314, and a head 320. In some embodiments, the insertion tool 310 includes a torque transfer protrusion element 2345. In some embodiments, the torque transfer protrusion 2345 can interact or engage or fit within the slots 333 of sliding element 335 (see FIG. 3). In some embodiments, the insertion tool includes control pins 312. In some embodiments, the insertion tool 310 includes 1 or more pins. In some embodiments, the insertion tool 310 includes 3 pins. In some embodiments, the control pins 312 are removable. In some embodiments, the controls pins 312 are fixed to the insertion tool 310. In some embodiments, the insertion tool 310 includes a cannulated region 317 (not shown in FIG. 1, see FIG. 2). In some embodiments, the insertion tool 310 comprises an aperture 315 at the second end 314 configured to receive a connection bolt, such as connection bolt 303.

In one or more embodiments, the compression device 3005 includes a threaded body 351, a sliding element 335, a compression element 340, and a setscrew 345.

In one or more embodiments, the insertion tool 310 is connected to the compression device 3005 via the plurality of control pins 312 inserted into a first end 316 of the insertion tool 310 and further inserted through voids of the sliding element 335. In various embodiments, stretching the compression element 340 includes securing a position of the sliding element 335 while applying a force to the threaded body 351 via the plurality of control pins 312. In at least one embodiment, the applied force causes the threaded body 351 to translate away from the stationary sliding element 335, thereby causing the compression element 340 to stretch. According to one embodiment, to secure the stretched/deformed state of the compression element 340, a connection bolt 303 is inserted through a second end 314 of the insertion tool 310 and further inserted into the sliding element 335. In at least one embodiment, the head 302 (which may be in the form of a nail or screw head) prevents further insertion of the connection bolt 303 into the compression assembly 3005. In various embodiments, the connection bolt 303 is rotated to connect to the sliding element 335 by an interface of the threads located on connection mechanism 306. According to one embodiment, upon being connected to the sliding element 335, the connection bolt 303 prevents the sliding element 335 from translating toward the threaded body, thereby preserving the stretched/deformed state of the compression element 340.

In some embodiments, the sliding element 335 includes a sliding element cap, a shaft 334, a first end 336, a second end 337, and internal threads 339 (not shown in FIG. 1, see FIG. 3). In some embodiments, the sliding element 335 includes slots 333 configured to receive control pins 312 attached to insertion tool 310. In some embodiments, the slots 333 can be additionally configured to receive a torque transfer protrusion element 2345. In some embodiments, engagement of the torque transfer protrusion 2345 provides enhanced torque transfer between the insertion tool 310 and the sliding element 335. In some embodiments, the slots 333 are configured to receive a variety of shapes of control pins 312.

In some embodiments, the compression device assembly 3005 comprises a setscrew 345. In some embodiments, the setscrew 345 is configured to maintain a position and orientation of compression element 340. In some embodiments, the setscrew 345 may interface with the connection bolt 303. For instance, the connection bolt 303 may interact with the setscrew by providing additional support via contact to setscrew 345 to maintain compression of compression element 340. In some embodiments, the setscrew 345 comprises threads that engage with the internal threads 339 of the sliding element 335. In some embodiments, the setscrew 345 comprises a biocompatible material. For example, the setscrew 345 may comprise an implant-grade metal, such as titanium or a titanium alloy, stainless steel, or a cobalt-chromium alloy. In other embodiments, the setscrew 345 may comprise a polymeric material, such as polyether ether ketone (PEEK). In some embodiments, the setscrew 345 is formed of a material selected to inhibit rotation and/or backing out of the compression element 340 during use.

In some embodiments, the compression device assembly 3005 includes a compression element 340. In some embodiments, the compression element 340 comprises a first end 324 and a second end 328. In some embodiments, the compression element 340 comprises a shaft 341. In some embodiments, the compression element 340 comprises a cannulated arrow tip 325. In some embodiments, the compression element 340 is cannulated and/or has a hollow interior. In other embodiments, the compression element 340 is not cannulated and/or does not have a hollow interior. In some embodiments, compression element 340 includes a slotted end 828. In some embodiments, the slotted end 828 is configured with a slot configured to receive an insert. In some embodiments, the insert could be a screwdriver used to allow for assembly of the compression element 340.

In some embodiments, the compression element 340 comprises a connection mechanism 326 at second end 328. In some embodiments, the connection mechanism 326 comprises threads configured to interface with the internal threads 339 of the sliding element 335. In some examples, the connection mechanism 326 comprises threads configured to interface with the setscrew 345.

According to one embodiment, the threaded body 351 includes a first end 358 and a second end 359. In various embodiments, the threaded body includes a tip 3006 at the first end 358. In various embodiments, the threaded body includes a shaft 352 (not shown in FIG. 1, see FIG. 3) between the second end 359 and the end of tip 3006. In some embodiments, the threaded body comprises an opening 370 at the first end 358. In one or more embodiments, the tip 3006 is integrally formed with the shaft 352. In some embodiments, the threaded body 351 includes a cannulated region 353. According to one embodiment, the shaft 352 and the tip 3006 are connected by one or more mechanisms including, but not limited to, threaded fittings, adhesives, welds, friction fits, and other connection mechanisms.

In at least one embodiment, the tip 3006 is configured to penetrate into biological material, such as bone or a tendon, at a target site. In one or more embodiments, the tip includes threads 354 and one or more blades 355 for drilling into material, such as bone or a tendon, via rotation of the compression device 3005. In various embodiments, the threads 354 interface with tissue, such as bone, to secure an implanted position of the compression device 3005 and resist pullout and pull-through forces experienced thereby.

In some embodiments, the threaded body 351 further comprises an aperture 360 (not shown in FIG. 1, see FIG. 3). In some embodiments, the aperture 360 is configured to receive sliding element 335. In some embodiments, the aperture 360 is configured to interact with a first portion 872 of sliding element 335.

FIG. 2 shows a cross-sectional view of an example of compression assembly 3004, according to one embodiment of the present disclosure.

FIG. 3 is a perspective view of the components of an exemplary compression device assembly 3004 according to one embodiment of the present disclosure.

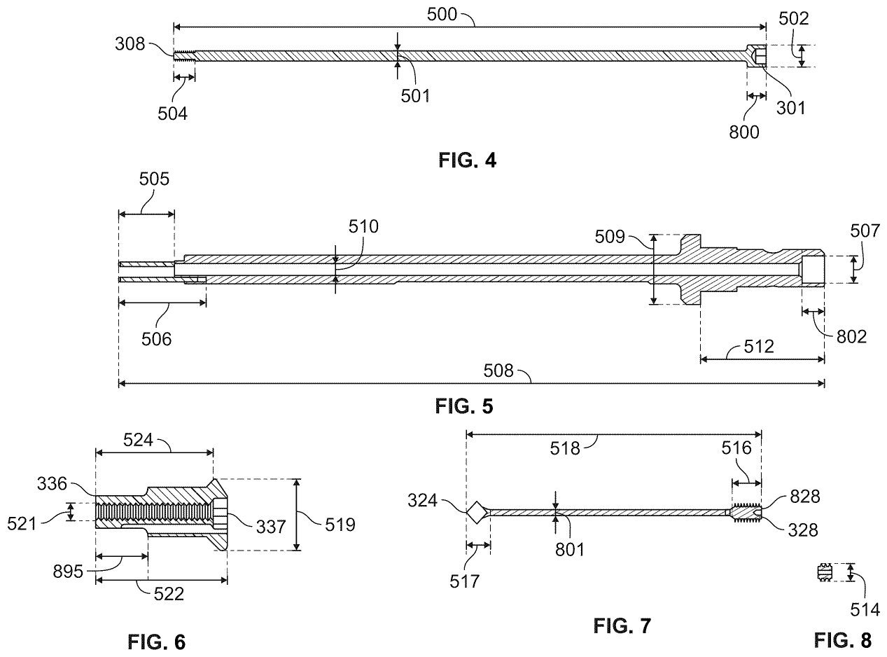

FIG. 4 shows a cross-section of connection bolt 303. In one or more embodiments, connection bolt 303 includes a total length 500 that measures about 20-160 mm between first end 308 and second end 301. In some embodiments, length 500 measures about 20-60 mm, 60-90 mm, 90-120 mm, or 120-160 mm. In some embodiments, the connection mechanism 306 includes a length 504 that measures about 0.5-50 mm. In some embodiments, the connection mechanism 306 includes a length 504 that measures about 0.5-12.9 mm, 12.9-25.25 mm, 25.25-37.6 mm, or 37.6-50 mm. In some embodiments, the head 302 includes a length 800 that measures about 0.5-50 mm. In some embodiments, the head 302 includes a length 800 that measures about 0.5-12.9 mm, 12.9-25.25 mm, 25.25-37.6 mm, or 37.6-50 mm.

In some embodiments, the head 302 has a diameter 502 that measures about 3-10 mm. In some embodiments, the head 302 has a diameter 502 that measures about 3-5 mm, 5-6 mm, 6-8 mm, or 8-10 mm.

In some embodiments, the shaft has a diameter 501 that measures about 1-9 mm. In some embodiments, the shaft has a diameter 501 that measures about 1-3 mm, 3-5 mm, 5-7 mm, or 7-9 mm.

FIG. 5 shows a cross-section of insertion tool 310. In one or more embodiments, the insertion tool 310 includes a total length 508 that measures about 15 to 175 mm between the end of control pin 312 and the first end 316. In some embodiments, the insertion tool 310 includes a length 508 that measures about 15-175 mm. In some embodiments, the insertion tool 310 includes a length 508 that measures about 15-60 mm, 60-100 mm, 100-140 mm, or 140-175 mm. In some embodiments, the insertion tool 310 includes a length 512 that measures about 3-100 mm. In some embodiments, the insertion tool 310 includes a length 512 that measures about 3-30 mm, 30-50 mm, 50-80 mm, or 80-100 mm. In some embodiments, the control pin 312 includes a length 506 that measures about 0.05-25 mm. In some embodiments, the control pin 312 includes a length 506 that measures about 0.05-6.3 mm, 6.3-12.5 mm, 12.5-18.75 mm, or 18.75-25 mm. In some embodiments, the length first end 316 to end of control pin 312 includes a length 505 that measures about 0.03-20 mm. In some embodiments, the length first end 316 to end of control pin 312 includes a length 505 that measures about 0.03-5.0 mm, 5.0-10.0 mm, 10.0-15.0 mm, or 15.0-20 mm. In some embodiments, the head 320 includes a length 802 that measures about 0.3-15 mm. In some embodiments, the head 320 includes a length 802 that measures about 0.3-3.95 mm, 3.95-7.65 mm, 7.65-11.3 mm, or 11.3-15 mm.

In some embodiments, the insertion tool 310 has a diameter 509 that measures about 5-50 mm. In some embodiments, the insertion tool 310 has a diameter 509 that measures about 5-16 mm, 16-28 mm, 28-39 mm, or 39-50 mm. In some embodiments, the head 320 has a diameter 507 that measures about 2-45 mm. In some embodiments, the head 320 has a diameter 507 that measures about 2-13 mm, 13-24 mm, 24-34 mm, or 34-45 mm. In some embodiments, the cannulated region 317 has a diameter 510 that measures about 0.05-20 mm. In some embodiments, the cannulated region 317 has a diameter 510 that measures about 0.05-5 mm, 5-10 mm, 10-15 mm, or 15-20 mm.

FIG. 6 shows a cross-section of sliding element 335. In some embodiments, sliding element 335 comprises a total length 522 from first end 336 to second end 337 that measures about 3 mm to 25 mm. In some embodiments, length 522 measures about 3-8 mm, 8-14 mm, 14-20 mm, or 20-25 mm. In some embodiments, the internal threads 339 include a length 524 that measures about 2-24 mm. In some embodiments, the internal threads 339 include a length 524 that measures about 2-8 mm, 8-13 mm, 13-18 mm, or 18-24 mm. In some embodiments, the first portion 872 includes a length 895 that measures about 0.03-15 mm. In some embodiments, the first portion 872 includes a length 895 that measures about 0.03-3.5 mm, 3.5-7.5 mm, 7.5-11.25 mm, or 11.25-15 mm.

In some embodiments, the sliding element 335 has a diameter 519 that measures about 0.25-15 mm. In some embodiments, the sliding element 335 has a diameter 519 that measures about 0.25-4.0 mm, 4.0-7.5 mm, 7.5-11.5 mm, or 11.5-15 mm. In some embodiments, the internal threads 339 have a diameter 521 that measures about 0.05-10 mm. In some embodiments, the internal threads 339 have a diameter 521 that measures about 0.05-2.55 mm, 2.55-5 mm, 5 -7.5 mm, or 7.5-10 mm.

FIG. 7 shows a cross-section of compression element 340. In various embodiments, the compression element 340 generates compressive loads (e.g., via application of tensile forces) that measure about 25-850 Newtons (N), about 25-100 N, about 100-150 N, about 150-200 N, about 200-250 N, about 250-300 N, about 300-350 N, about 350-400 N, about 400-450 N, about 450-500 N, about 500-550 N, about 550-600 N, about 600-650 N, about 650-700 N, about 700-750 N, about 750-800 N, or about 800-850 N.

In some embodiments, the compression element 340 comprises a total length 518 from first end 324 to second end 328 that measures about 2 mm to 16 0 mm. In some embodiments, length 518 measures about 2-40 mm, 40-80 mm, 80-120 mm, or 120-160 mm. In some embodiments, the connection mechanism 326 includes a length 516 that measures about 0.05-50 mm. In some embodiments, the connection mechanism 326 includes a length 516 that measures about 0.05-12.5 mm, 12.5-25 mm, 25-37.5 mm, or 37.5-50 mm. In some embodiments, the cannulated arrow tip 325 includes a length 517 that measures about 0.04-40 mm. In some embodiments, the cannulated arrow tip 325 includes a length 517 that measures about 0.04-10.0 mm, 10.0-20.0 mm, 20.0-30.0 mm, or 30.0-40 mm. In some embodiments, the compression element 340 has a diameter 801 that measures about 0.01-20 mm. In some embodiments, the compression element 340 has a diameter 801 that measures about 0.01-5.01 mm, 5.01-10 mm, 10-15 mm, or 15-20 mm. In some embodiments, the compression element 340 has a diameter that is configured to fit within the threaded body 351.

FIG. 8 shows a cross-sectional view of setscrew 345. In some embodiments, setscrew 345 comprises a length 514 that measures about 0.05 to 5 mm. In some embodiments, the setscrew 345 includes a length 514 that measures about 0.05-1.3 mm, 1.3-2.5 mm, 2.5-3.75 mm, or 3.75-5 mm.

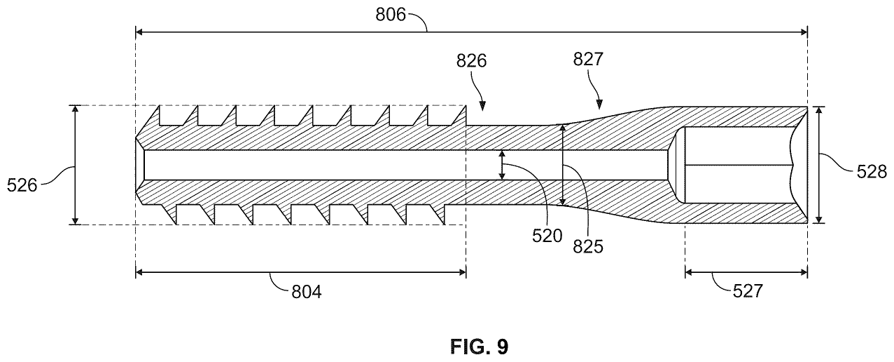

FIG. 9 provides a cross-sectional view of threaded body 351. In some embodiments, the threaded body 351 includes a total length 806 between the first end 358 and the second end 359 that measures about 5 mm to 160 mm. In some embodiments, length 806 measures about 5-40 mm, 40-80 mm, 80-120 mm, or 120-160 mm. In some embodiments, the threads 354 include a length 804 that measures about 1-100 mm. In some embodiments, the threads 354 include a length 804 that measures about 1-30 mm, 30-50 mm, 50-80 mm, or 80-100 mm. In some embodiments, the aperture 360 includes a length 527 that measures about 0.5-75 mm. In some embodiments, the aperture 360 includes a length 527 that measures about 0.5-20 mm, 20-38 mm, 38-55 mm, or 55-75 mm.

In some embodiments, the threads 354 have a diameter 526 that measures about 0.5-15 mm. In some embodiments, the threads 354 have a diameter 526 that measures about 0.5-4 mm, 4.0-7.75 mm, 7.75-11.5 mm, or 11.5-15 mm. In some embodiments, the cannulated region 353 has a diameter 520 that measures about 0.01-10 mm.

In some embodiments, the cannulated region 353 has a diameter 520 that measures about 0.01-2.5 mm, 2.5-5 mm, 5 -7.5 mm, or 7.5-10 mm.

In some embodiments, the threaded body 351 has a diameter 825 that measures about 0.1-13 mm. In some embodiments, the threaded body 351 has a diameter 825 that measures about 0.1-3.3 mm, 3.3-6.55 mm, 6.55-10.0 mm, or 10-13 mm.

In some embodiments, threaded body 351 comprises a first portion 826 and a second portion 827. In some embodiments, the threaded body 351 includes a tapered transition region in which the outer diameter varies along the longitudinal axis in a multi-stage manner, such that a first portion 826 defines an initial tapered segment that begins at (or near) a reduced-diameter region and transitions toward a larger outer diameter, and a second portion 827 defines a subsequent tapered segment adjoining the first portion 826 that continues (and/or completes) the transition to the relatively larger-diameter region adjacent the proximal end of the threaded body 351, thereby providing a compound taper formed by two sequential taper portions rather than a single uniform step.

FIG. 10 is a side view 3000 of components of an exemplary compression device assembly 3004 according to one embodiment of the present disclosure.

FIG. 11 is an alternative side view 3001 of components of an exemplary compression device assembly 3004 according to one embodiment of the present disclosure.

FIG. 12 is a perspective view 3002 of the components of an exemplary compression device assembly 3004 according to one embodiment of the present disclosure.

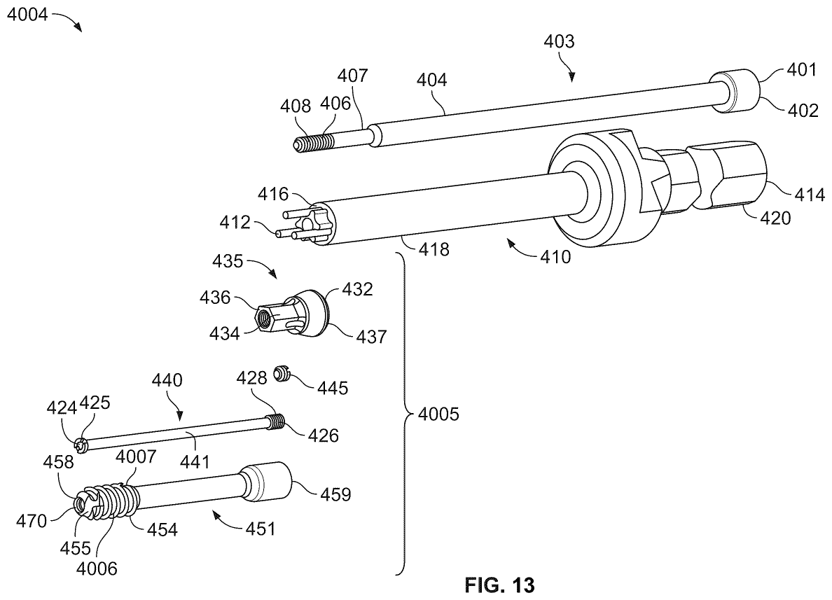

FIG. 13, which shows a perspective view of an exemplary compression assembly 4004 according to various embodiments of the present disclosure. As will be understood and appreciated, the exemplary compression assembly 4004 shown in FIG. 13 represents merely one approach or embodiment of the present system, and other aspects are used according to various embodiments of the present system.

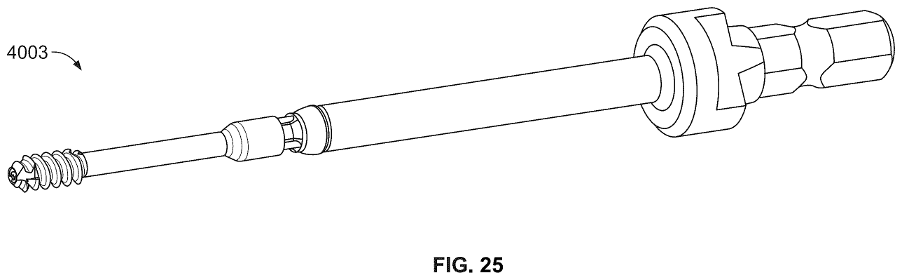

In various embodiments, the assembly 4004 includes a compression device 4005, an insertion tool 410, and a connection bolt 403. In some embodiments, the compression device 4005, the insertion tool 410, and the connection bolt 403 are provided separately (e.g., unattached or unassembled), such as, for example, in a kit. According to one embodiment, the compression device 4005 and insertion tool 410 are provided as shown in FIG. 13 or FIG. 25, and the compression device 4005 is provided stretched according to predetermined parameters. In alternate embodiments, the compression device 4005 and insertion tool 410 are provided as the assembly 4004, but the compression device 4005 is not stretched (e.g., the stretching being performed by a user, such as a technician or surgeon). In some embodiments, the compression device 4005 is insertable without the insertion tool 410.

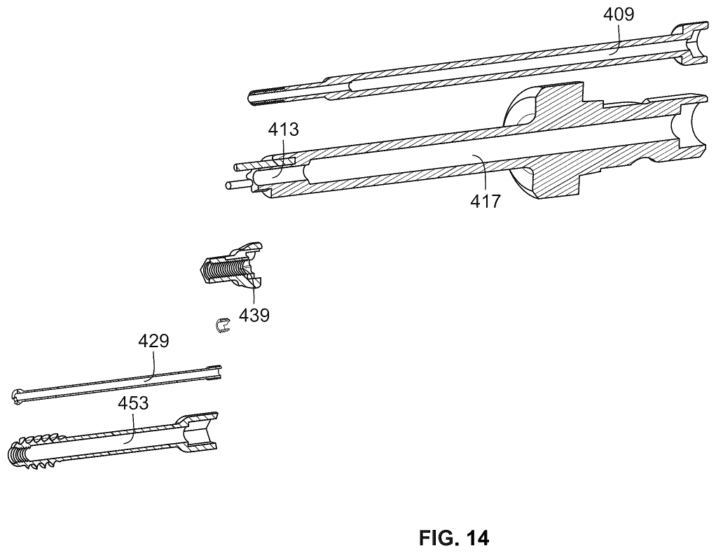

In various embodiments, the connection bolt 403 comprises a second end 401, a head 402, a shaft 404, a connection mechanism 406, and a first end 408. In some embodiments, the connection mechanism 406 is configured to interface with the sliding element 435. In some embodiments, the connection mechanism 406 comprises threads configured to interface with the internal threads 439 of the sliding element 435. In some examples, the connection bolt 403 comprises a first end body 407 configured to be received by a first end inner boundary 413 of insertion tool 410 (see FIG. 14). In some examples, the connection bolt 403 comprises a cannulated region 409 (not shown in FIG. 13, see FIG. 14).

According to one embodiment, the connection bolt 403 comprises a substantially cylindrical shape. In other embodiments, the connection bolt 403 comprises one or more shapes including, but not limited to circles, semi-circles, hexagons, and other polygons. In some embodiments, the connection bolt 403 is cannulated and/or comprises a hollow interior. In some embodiments, the connection bolt 403 is not cannulated and/or does not comprise a hollow interior. In some embodiments, the connection mechanism 406 comprises threads.

According to one embodiment, the connection bolt 403 is provided in a kit (e.g., including the components of the compression assembly 4004) and a user inserts and rotates the connection bolt 403 to achieve a desired tensioning of the compression device 4005. In at least one embodiment, the connection bolt 403 is inserted into the compression assembly 4004 during an assembly process. In one or more embodiments, the tensioning of the compression element 440 is performed according to predetermined implantation parameters, for example, a desired compression force to be applied to skeletal elements of a patient.

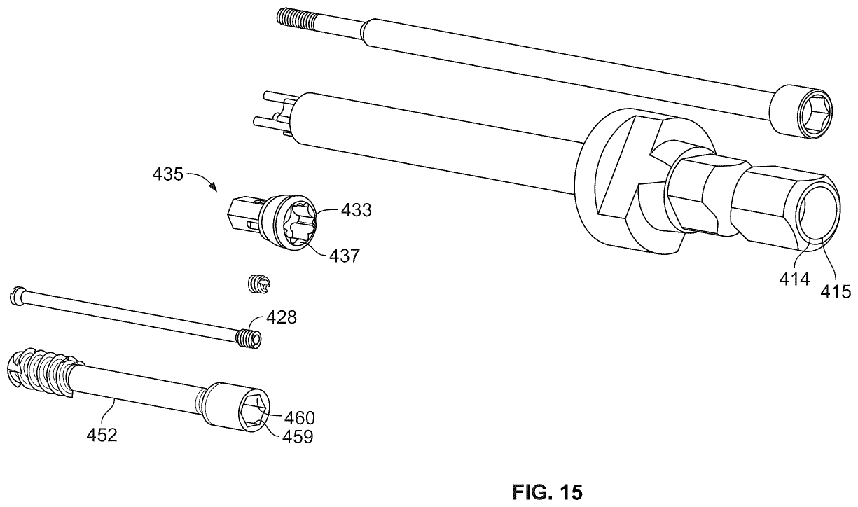

In one or more embodiments, the insertion tool 410 includes a first end 416, a second end 414, and a head 420. In some embodiments, the insertion tool 410 includes a torque transfer protrusion element 3345. In some embodiments, the torque transfer protrusion 3345 can interact or engage or fit within the slots 433 of sliding element 435 (see FIG. 15). In some embodiments, the insertion tool 410 includes control pins 412. In some embodiments, the insertion tool 410 includes 1 or more pins. In some embodiments, the insertion tool 410 includes 3 pins. In some embodiments, the control pins 412 are removable. In some embodiments, the controls pins 412 are fixed to the insertion tool. In some embodiments, the insertion tool 410 includes a cannulated region 417 (not shown in FIG. 13, see FIG. 14). In some embodiments, the insertion tool comprises an aperture 415 (see FIG. 15) at the second end 414 configured to receive a connection bolt, such as connection bolt 403.

In one or more embodiments, the compression device 4005 includes a threaded body 451, a sliding element 435, a compression element 440, and a setscrew 445.

In one or more embodiments, the insertion tool 410 is connected to the compression device 4005 via the plurality of control pins 412 inserted into a first end 416 of the insertion tool 410 and further inserted through voids of the sliding element 435. In various embodiments, stretching the compression element 440 includes securing a position of the sliding element 435 while applying a force to the threaded body 451 via the plurality of control pins 412. In at least one embodiment, the applied force causes the threaded body 451 to translate away from the stationary sliding element 435, thereby causing the compression element 440 to stretch. According to one embodiment, to secure the stretched/deformed state of the compression element 440, a connection bolt 403 is inserted through a second end 414 of the insertion tool 410 and further inserted into the sliding element 435. In at least one embodiment, the head 402 (which may be in the form of a nail or screw head) prevents further insertion of the connection bolt 403 into the compression assembly 4005. In various embodiments, the connection bolt 403 is rotated to connect to the sliding element 435 by an interface of the threads located on connection mechanism 406. According to one embodiment, upon being connected to the sliding element 435, the connection bolt 403 prevents the sliding element 435 from translating toward the threaded body, thereby preserving the stretched/deformed state of the compression element 440.

In some embodiments, the sliding element 435 includes a sliding element cap, a shaft 434, a first end 436, a second end 437, and internal threads 439 (not shown in FIG. 13, see FIG. 14). In some embodiments, the sliding element 435 includes slots 433 configured to receive control pins 412 attached to insertion tool 410. In some embodiments, the slots 433 can be additionally configured to receive a torque transfer protrusion element 3345. In some embodiments, engagement of the torque transfer protrusion 3345 provides enhanced torque transfer between the insertion tool 410 and the sliding element 435. In some embodiments, the slots 433 are configured to receive a variety of shapes of control pins 412.

In some embodiments, the compression device assembly 4005 comprises a setscrew 445. In some embodiments, the setscrew 445 is configured to maintain a position and orientation of compression element 440. In some embodiments, the setscrew 445 may interface with the connection bolt 403. In some embodiments, the setscrew 445 prevents the compressive element 440 from rotating. In some embodiments, the setscrew 445 prevents the compressive element 440 from transversing along the sliding element. For instance, the connection bolt 403 may interact with the setscrew by providing additional support via contact to setscrew 445 to maintain compression of compression element 440. In some embodiments, the setscrew 445 comprises threads that engage with the internal threads 439 of the sliding element 435. In some embodiments, the setscrew 445 comprises a biocompatible material. For example, the setscrew 445 may comprise an implant-grade metal, such as titanium or a titanium alloy, stainless steel, or a cobalt-chromium alloy. In other embodiments, the setscrew 445 may comprise a polymeric material, such as polyether ether ketone (PEEK). In some embodiments, the setscrew 445 is formed of a material selected to inhibit rotation and/or backing out of the compression element 440 during use.

In some embodiments, the compression device assembly 4005 includes a compression element 440. In some embodiments, the compression element 440 comprises a first end 424 and a second end 428. In some embodiments, the compression element 440 comprises a shaft 441. In some embodiments, the compression element 440 comprises a collar 425 at first end 424. In some embodiments, the compression element 440 is cannulated and/or has a hollow interior. In other embodiments, the compression element 440 is not cannulated and/or does not have a hollow interior.

In some embodiments, the compression element 440 comprises a connection mechanism 426 at second end 428. In some embodiments, the connection mechanism 426 comprises threads configured to interface with the internal threads 439 of the sliding element 435. In some examples, the connection mechanism 426 comprises threads configured to interface with the setscrew 445.

According to one embodiment, the threaded body 451 includes a first end 458 and a second end 459. In various embodiments, the threaded body includes a tip 4006 at the first end 458. In various embodiments, the threaded body includes a shaft 452 (not shown in FIG. 13, see FIG. 15) between the second end 459 and a tip end 4007 (e.g. end of the tip 4006). In some embodiments, the threaded body comprises an opening 470 at the first end 458. In one or more embodiments, the tip 4006 is integrally formed with the shaft 452. According to one embodiment, the shaft 452 and the tip 4006 are connected by one or more mechanisms including, but not limited to, threaded fittings, adhesives, welds, friction fits, and other connection mechanisms.

In at least one embodiment, the tip 4006 is configured to penetrate into biological material, such as bone or a tendon, at a target site. In one or more embodiments, the tip includes threads 454 and one or more blades 455 for drilling into material, such as bone or a tendon, via rotation of the compression device 4005. In various embodiments, the threads 454 interface with tissue, such as bone, to secure an implanted position of the compression device 4005 and resist pullout and pull-through forces experienced thereby.

In some embodiments, the threaded body 451 further comprises an aperture 460 (not shown in FIG. 13, see FIG. 15). In some embodiments, the aperture 460 is configured to receive sliding element 435. In some embodiments, the aperture 460 is configured to interact with the shaft 434 of sliding element 435.

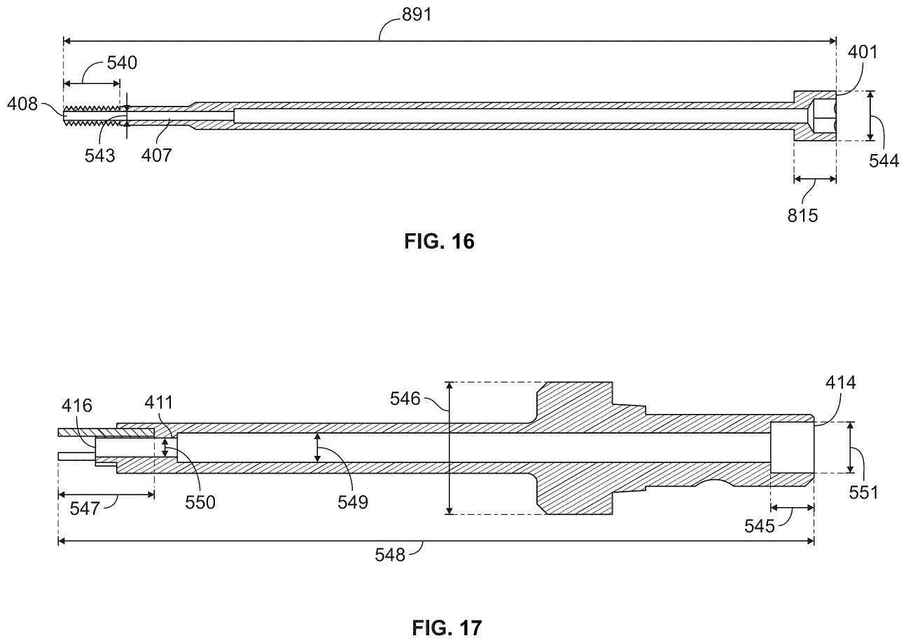

FIG. 16 shows a cross-section of connection bolt 403. In one or more embodiments, connection bolt 403 includes a total length 891 that measures about 20-200 mm between first end 408 and second end 401. In some embodiments, length 891 measures about 20-40 mm, 40-60 mm, 60-80 mm, or 80-100 mm, 100-120 mm, 120-140 mm, 140-160 mm, 160-180 mm, or 180-200 mm. In some embodiments, the head 402 includes a length 815 that measures about 1-50 mm. In some embodiments, the head 402 includes a length 815 that measures about 1-13 mm, 13-26 mm, 26-38 mm, or 38-50 mm. In some embodiments, the connection mechanism 406 includes a length 540 that measures about 2-45 mm. In some embodiments, the connection mechanism 406 includes a length 540 that measures about 2-13 mm, 13-24 mm, 24-34 mm, or 34-45 mm. In some embodiments, the head 402 has a diameter 544 that measures about 10-50 mm. In some embodiments, the head 402 has a diameter 544 that measures about 10-20 mm, 20-30 mm, 30-40 mm, or 40-50 mm. In some embodiments, the cannulated region 409 has a diameter of 0.05-20 mm. In some embodiments the cannulated region 409 has a diameter of 0.05-20 mm, 0.05-5 mm, 5 mm-10 mm, or 10 mm-20 mm. In some embodiments, the first end body 407 diameter has a length of 553 that is less than the diameter of the diameter of the head 402, but greater than the diameter of the cannulated region 409.

FIG. 17 shows a cross-section of insertion tool 410. In one or more embodiments, the insertion tool 410 includes a total length 548 that measures about 15-200 mm between the end of control pin 412 and the first end 416. In some embodiments, length 548 measures about 20-60 mm, 60-110 mm, 110-150 mm, or 150-200 mm. In some embodiments, the head 420 includes a length 545 that measures about 1-50 mm. In some embodiments, the head 420 includes a length 545 that measures about 1-13 mm, 13-26 mm, 26-38 mm, or 38-50 mm. In some embodiments, the control pin 412 includes a length 547 that measures about 0.05-25 mm. In some embodiments, the control pin 412 includes a length 547 that measures about 0.05-6.3 mm, 6.3-12.5 mm, 12.5-18.75 mm, or 18.75-25 mm. In some embodiments, the length first end 416 to end of control pin 412 includes a length that measures about 0.05-20 mm. In some embodiments, the length first end 416 to end of control pin 412 includes a length that measures about 0.05-5.05 mm, 5.05-10 mm, 10-15 mm, or 15-20 mm. In some embodiments, the insertion tool 410 has a diameter 546 that measures about 5-50 mm.

In some embodiments, the insertion tool 410 has a diameter 546 that measures about 5-16 mm, 16-28 mm, 28-39 mm, or 39-50 mm. In some embodiments, the head 420 has a diameter 551 that measures about 0.1-25 mm. In some embodiments, the head 420 has a diameter 551 that measures about 0.1-6.3 mm, 6.3-12.55 mm, 12.55-18.75 mm, or 18.75-25 mm. In some embodiments, the cannulated region 417 has a diameter 549 that measures about 0.5-15 mm. In some embodiments, the cannulated region 417 has a diameter 549 that measures about 0.5-4.1 mm, 4.1-7.75 mm, 7.75-11.4 mm, or 11.4-15 mm. In some embodiments, the first end inner boundary 413 has a diameter 550 that measures about 0.1-15 mm. In some embodiments, the first end inner boundary 413 has a diameter 550 that measures about 0.1-3.8 mm, 3.8-7.5 mm, 7.5-11.5 mm, or 11.5-15 mm.

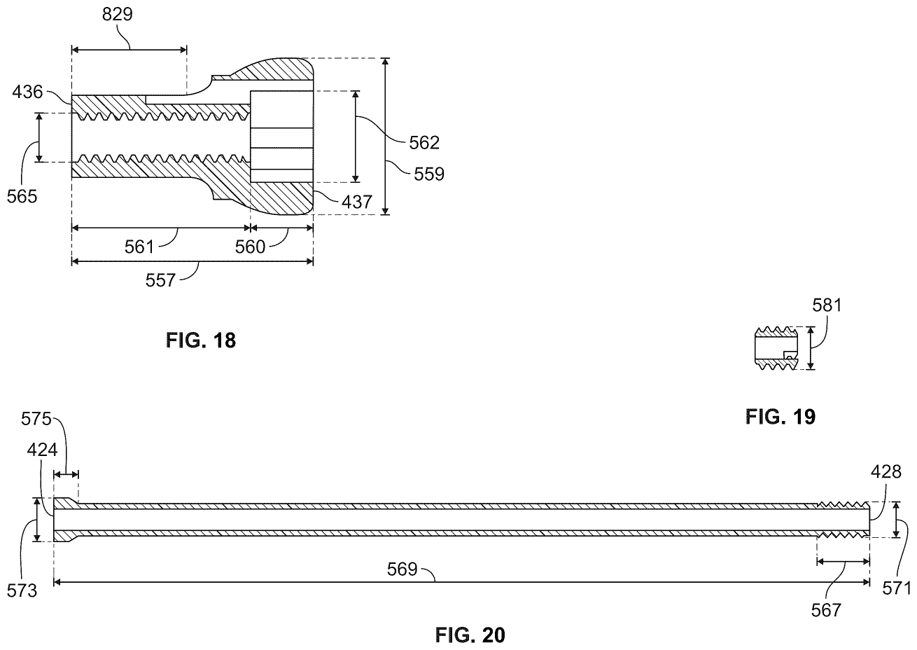

FIG. 18 shows a cross-section of sliding element 435. In some embodiments, sliding element 435 comprises a total length 557 from first end 436 to second end 437 that measures about 3 mm to 50 mm. In some embodiments, length 557 measures about 3-15 mm, 15-26 mm, 26-38 mm, or 38-50 mm. In some embodiments, the internal threads 439 include a length 561 that measures about 0.25-45 mm. In some embodiments, the internal threads 439 include a length 561 that measures about 0.25-11.45 mm, 11.45-22.6 mm, 22.6-33.8 mm, or 33.8-45 mm. In some embodiments, the slots 433 includes a length 560 that measures about 0.2-15 mm. In some embodiments, the slots 433 includes a length 560 that measures about 0.2-3.9 mm, 3.9-7.6 mm, 7.6-11.3 mm, or 11.3-15 mm. In some embodiments, the Sliding Element 435 includes a length 829 that measures about 0.05-10 mm. In some embodiments, the Sliding Element 435 includes a length 829 that measures about 0.05-2.55 mm, 2.55-5 mm, 5 -7.5 mm, or 7.5-10 mm.

In some embodiments, the sliding element 435 has a diameter 559 that measures about 0.25-15 mm. In some embodiments, the sliding element 435 has a diameter 559 that measures about 0.25-3.95 mm, 3.95-7.6 mm, 7.6-11.3 mm, or 11.3-15 mm.

In some embodiments, the slot 433 has a diameter 562 that measures about 0.005-5 mm. In some embodiments, the slot 433 has a diameter 437 that measures about 0.01-1.25 mm, 1.25-2.5 mm, 2.5-3.75 mm, or 3.75-5 mm. In some embodiments, the internal threads 439 has a diameter 565 that measures about 0.05-5 mm. In some embodiments, the internal threads 439 has a diameter 565 that measures about 0.05-1.3 mm, 1.3-2.5 mm, 2.5-3.75 mm, or 3.75-5 mm.

FIG. 19 shows a cross-sectional view of setscrew 445. In some embodiments, setscrew 445 comprises a length 581 that measures about 0.05 mm to 10 mm.

FIG. 20 shows a cross-section of compression element 440. In various embodiments, the compression element 440 generates compressive loads (e.g., via application of tensile forces) that measure about 25-850 Newtons (N), about 25-100 N, about 100-150 N, about 150-200 N, about 200-250 N, about 250-300 N, about 300-350 N, about 350-400 N, about 400-450 N, about 450-500 N, about 500-550 N, about 550-600 N, about 600-650 N, about 650-700 N, about 700-750 N, about 750-800 N, or about 800-850 N.

In some embodiments, the compression element 440 comprises a total length 569 from first end 424 to second end 428 that measures about 2 mm to 160 mm. In some embodiments, length 569 measures about 2-40 mm, 40-80 mm, 80-120 mm, or 120-160 mm. In some embodiments, the connection mechanism 426 includes a length 567 that measures about 0.05-50 mm. In some embodiments, the connection mechanism 426 includes a length 567 that measures about 0.05-12.55 mm, 12.55-25 mm, 25-37.5 mm, or 37.5-50 mm. In some embodiments, the connection mechanism collar 425 includes a length 575 that measures about 0.02-40 mm. In some embodiments, the connection mechanism collar 425 includes a length 575 that measures about 0.02-10.01 mm, 10.01-20.01 mm, 20.01-30 mm, or 30-40 mm. In some embodiments, the connection mechanism 426 has a diameter 571 that measures about 0.5-9.5 mm. In some embodiments, the connection mechanism 426 has a diameter 571 that measures about 0.5-2.75 mm, 2.75-5 mm, 5 -7.25 mm, or 7.25-9.5 mm. In some embodiments, the connection mechanism collar 425 has a diameter 573 that measures about 0.5-10 mm. In some embodiments, the connection mechanism collar 425 has a diameter 573 that measures about 0.5-2.9 mm, 2.9-5.25 mm, 5.25-7.6 mm, or 7.6-10 mm.

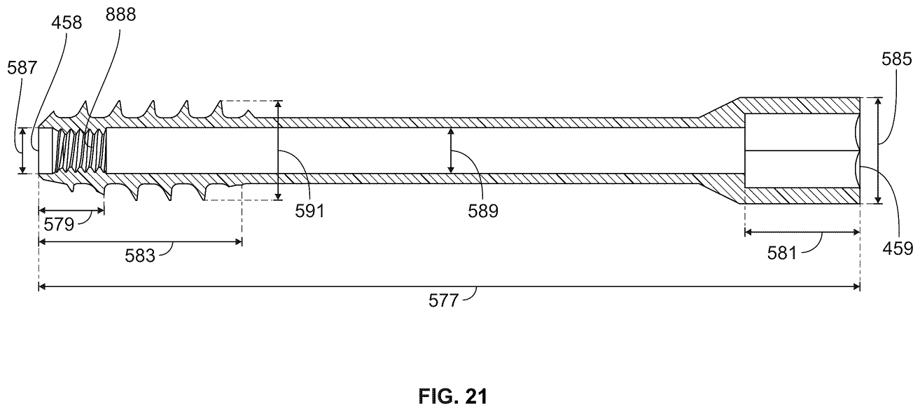

FIG. 21 provides a cross-sectional view of threaded body 451. In some embodiments, the threaded body 451 includes a total length 577 between the first end 458 and the second end 459 that measures about 5 mm to 160 mm. In some embodiments, length 577 measures about 5-40 mm, 40-80 mm, 80-120 mm, or 120-160 mm. In some embodiments, the threads 454 include a length 583 that measures about 0.5-50 mm. In some embodiments, the threads 454 include a length 583 that measures about 0.5-12.9 mm, 12.9-25.25 mm, 25.25-37.6 mm, or 37.6-50 mm. In some embodiments, the aperture 460 includes a length 581 that measures about 0.1-40 mm. In some embodiments, the aperture 460 includes a length 581 that measures about 0.1-10.05 mm, 10.05-20.05 mm, 20.05-30 mm, or 30-40 mm. In some embodiments, the internal threads 888 include a length 579 that measures about 0.05-30 mm. In some embodiments, the internal threads 888 include a length 579 that measures about 0.05-7.55 mm, 7.55-15 mm, 15-22.5 mm, or 22.5-30 mm. In some embodiments, the threaded body 451 has a diameter 585 that measures about 0.5-15 mm. In some embodiments, the threaded body 451 has a diameter 585 that measures about 0.5-4.1 mm, 4.1-7.75 mm, 7.75-11.4 mm, or 11.4-15 mm. In some embodiments, the threads 454 have a diameter 591 that measures about 0.4-15 mm. In some embodiments, the threads 454 have a diameter 591 that measures about 0.4-4.05 mm, 4.05-7.7 mm, 7.7-11.35 mm, or 11.35-15 mm. In some embodiments, the cannulated region 453 has a diameter 589 that measures about 0.3-14 mm.

In some embodiments, the cannulated region 453 has a diameter 589 that measures about 0.3-3.7 mm, 3.7-7.15 mm, 7.15-10.55 mm, or 10.55-14 mm. In some embodiments, the internal threads 888 has a diameter 587 that measures about 0.2-12 mm.

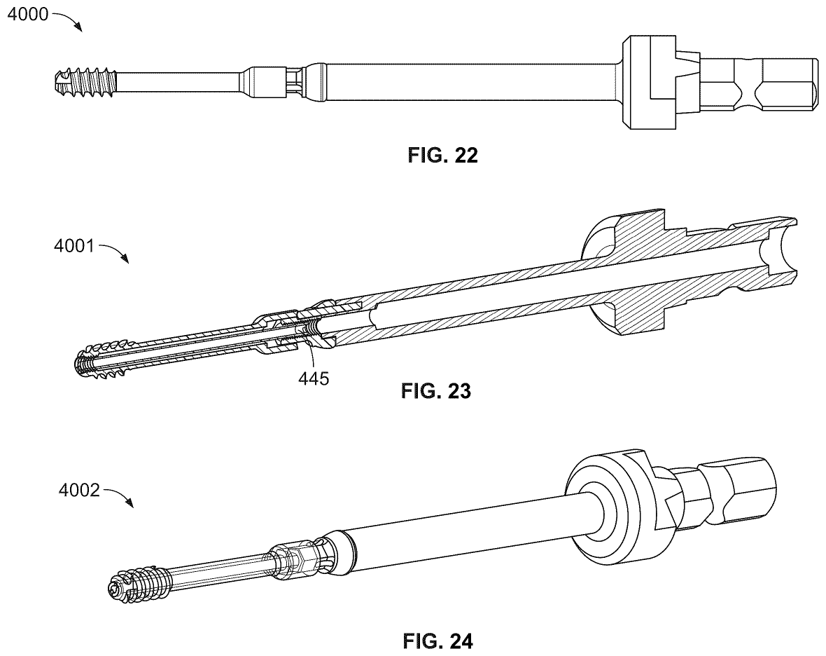

FIG. 22 is a side view 4000 of components of an exemplary compression device assembly 4004 according to one embodiment of the present disclosure.

FIG. 23 is a cross sectional view 4001 of components of an exemplary compression device assembly 4004 according to one embodiment of the present disclosure.

FIG. 24 is a perspective view 4002 of the components of an exemplary compression device assembly 4004 according to one embodiment of the present disclosure, wherein the threaded body 451 is transparent.

FIG. 25 is a perspective view 4003 of the components of an exemplary compression device assembly 4004 according to one embodiment of the present disclosure.

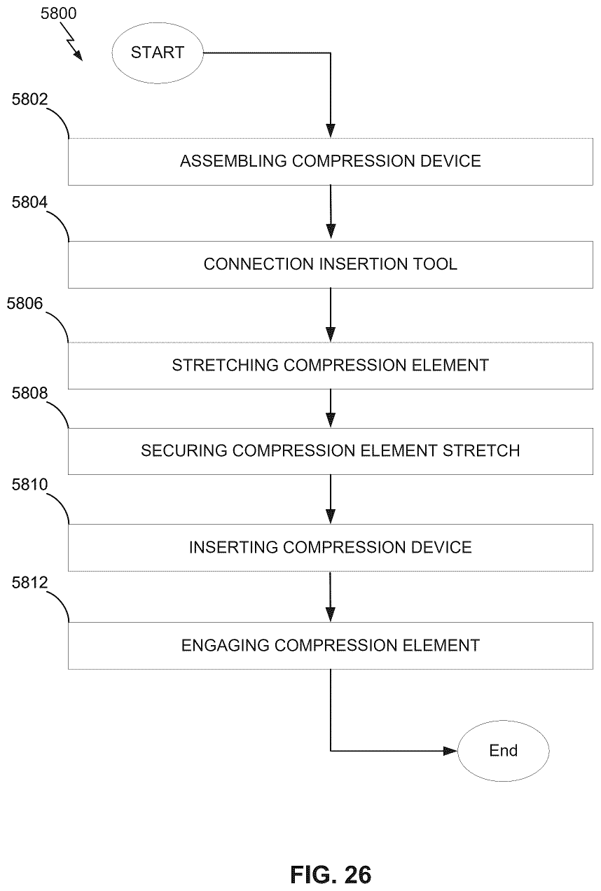

Before turning to the process flow diagrams of FIG. 26, it is noted that embodiments described herein may be practiced using an alternative order of the steps illustrated in FIG. 26. That is, the process flows illustrated in FIG. 26 are provided as examples only, and the embodiments may be practiced using process flows that differ from those illustrated. Additionally, it is noted that not all steps are required in every embodiment. In other words, one or more of the steps may be omitted or replaced, without departing from the spirit and scope of the embodiments. Further, steps may be performed in different orders, in parallel with one another, or omitted entirely, and/or certain additional steps may be performed without departing from the scope of the embodiments.

FIG. 26 shows an exemplary compression process 5800, according to one embodiment. At step 5802, the process 5800 includes assembling a compression device, such as any of compression devices 3005 (FIG. 1), or 4005 (FIG. 13). In at least one embodiment, assembling the compression device includes, but is not limited to, securing opposing ends of a compression element to a sliding element and a threaded body, respectively. In one example, the compression element, sliding element, and/or threaded body are rotated to engage threaded portions of each component and provide a secure connection. In another example, bayonet or luer-lock style fittings are engaged between each element to assemble the compression device. In some embodiments, the compression element is threaded into the threaded body and sliding element substantially simultaneously.

At step 5804, the process 5800 includes connecting the compression device to insertion tool, such as any insertion tools 310 (FIG. 1), or 410 (FIG. 13). In various embodiments, connecting the compression device to the insertion tool includes, but is not limited to, engaging a connection mechanism of the insertion tool with a corresponding receiver of the sliding element (e.g., by threading screw threads of the insertion tool into internal threads of the sliding element). In some embodiments, connecting the compression device to the insertion tool includes inserting a plurality of control pins through a plurality of voids or slots in the sliding element such that the plurality of control pins contact an end of the threaded body. In at least one embodiment, the plurality of voids or slots allow the compression device to translate along the plurality of control pins (e.g., in response to a force applied at either end of the compression device).

At step 5806, the process 5800 includes stretching the compression element. In at least one embodiment, stretching the compression element includes securing a stationary position of the sliding element while applying a force to an end of the threaded body, thereby causing the threaded body to translate away from the sliding element and resulting in the stretching of the compression element. In one example, the compression device and attached insertion tool are placed into a stretching device. In this example, a locking mechanism secures the stationary position of the sliding element. Continuing the example, after the sliding element is secured, a pushing mechanism applies a force to the insertion tool, and the force is translated to the end of the threaded body. In the same example, the force causes the threaded body to translate away from the sliding element, thereby stretching the compression element. In some embodiments, stretching of the compression element occurs during insertion of the compression device to a target site. In one example, rotation of a compression device into a target site causes the compression element to progressively stretch. In this example, the compression device is rotated into the target site until a predetermined level of stretch is achieved in the compression element.

At step 5808, the process 5800 includes securing the stretched position of the compression element. According to one embodiment, the stretched position is secured while the compression device and insertion tool are disposed within a stretching device. In at least one embodiment, securing the stretch of the compression element includes inserting a connection bolt through the insertion tool and into the sliding element, and securely attaching the connection bolt to the sliding element (e.g., in an impermanent manner such that the connection bolt may be detached via a tool). In one example, the connection bolt rotates into the sliding element such that corresponding threads on each component are engaged. In another example, rotating the inserted connection bolt engages a bayonet or luer-lock style fitting. In at least one embodiment, the connection bolt is inserted into the sliding element such that an end of the connection bolt contacts an end of the insertion tool, thereby preventing further insertion of the connection bolt. In various embodiments, upon release of the sliding element from the secured position within the stretching device, the driver or connection bolt prevents movement of the insertion tool, thereby preventing contraction of the compression element and preserving the stretched state of the same. In some embodiments, a setscrew is used to maintain a position and orientation of the compression element within the compression device assembly.

In some embodiments, steps 5802-5808 are performed as a first process at a first location (e.g., by a fabrication or assembly entity) and steps 5810-5812 are performed as a second process at a second location (e.g., by a surgeon or technician). In one example, a process for manufacturing a compression device includes steps 5802-5808 and a process for using the compression device includes steps 5810-5812.

At step 5810, the process 5800 includes inserting the compression device into a target site including at least a first and a second skeletal element to be compressed for the purposes of promoting healing, ossification, and/or fusion. In one example, a surgeon rotates the compression device (e.g., via manual or motorized rotation of the insertion tool) into a target site such that external threading of the sliding element lies in a first skeletal element and external threading of the threaded body lies in a second skeletal element. As will be understood from discussions herein, the sliding element may contact a skeletal element (or other tissue) in any suitable way. In one embodiment, the sliding element includes one or more external threads, such that the sliding element contacts a skeletal element via the one or more external threads drilling into or otherwise engaging with the skeletal element. In some embodiments, the sliding element may include a head (or other feature) such that a portion of the sliding element contacts a surface of a skeletal element (e.g., opposed to drilling into a skeletal element).

At step 5812, the process 5800 includes engaging the compression element such that a compressive force is generated between the first skeletal element and the second skeletal element. In one example, a surgeon disconnects the connection bolt from the sliding element, thereby causing the sliding element to attempt to translate toward the threaded body (e.g., in response to the tensile force applied by the compression element). In the same example, in response to tensile forces from the compression element, the sliding element applies a first sustained compressive force to the first skeletal element and the threaded body applies a second sustained compressive force to the second skeletal element (e.g., the first and second forces being applied in opposing directions). Continuing the example, the compressive forces promote ossification, resettling, and/or fusion between the first and second skeletal elements. In this example, whereas previous compression solutions may lose compressive force over time due to resettling and resorption of the skeletal elements, the compression element of the present compression device dynamically responds to movement and structural changes at the target site to maintain substantially continuous and constant compression of the first and second skeletal elements.

While various aspects have been described in the context of a preferred embodiment, additional aspects, features, and methodologies of the claimed assemblies, devices, and processes will be readily discernible from the description herein, by those of ordinary skill in the art. Many embodiments and adaptations of the disclosure and claimed assemblies, devices, and processes other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed assemblies, devices, and processes. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed assemblies, devices, and processes. In addition, some steps may be carried out simultaneously, contemporaneously, or in synchronization with other steps.

The embodiments were chosen and described in order to explain the principles of the claimed assemblies, devices, and processes and their practical application so as to enable others skilled in the art to utilize the assemblies, devices, and processes and various embodiments and with various modifications as are suited to the particular use contemplated. Alternative embodiments will become apparent to those skilled in the art to which the claimed assemblies, devices, and processes pertain without departing from their spirit and scope. Accordingly, the scope of the claimed assemblies, devices, and processes is defined by the appended claims rather than the foregoing description and the exemplary embodiments described therein.

We claim:

1. A compression device assembly comprising:
an elongate threaded body comprising:
a threaded body first end comprising one or more screw threads configured for affixing the threaded body to a first skeletal element of a patient and defining a hollow interior of the threaded body; and
a threaded body second end comprising an opening to the hollow interior of the threaded body;
a sliding element configured for contacting a second skeletal element of the patient, defining a hollow interior of the sliding element, and comprising:
a sliding element first end comprising an elongated portion shaped to interface with the opening of the threaded body second end and comprising one or more pin slots configured for receiving one or more pins of an insertion tool;
a sliding element second end defining one or more pin openings configured for receiving the one or more pins of the insertion tool, wherein the insertion tool defines a hollow interior of the insertion tool and comprises one or more pins configured to be received by the one or more pin openings and the one or more pin slots of the sliding element;
a setscrew configured to maintain a position and orientation of a nitinol compression element within the compression device assembly;
the nitinol compression element operatively connected to the threaded body and the sliding element, wherein the nitinol compression element is in a deformed state prior to insertion into the patient, wherein the compression device assembly is configured for applying compression to the first skeletal element and the second skeletal element at least in part via the nitinol compression element returning to a relaxed state from the deformed state.

2. The compression device assembly of claim 1, wherein the nitinol compression element further comprises a second end comprising a slotted end configured to receive an insert.

3. The compression device assembly of claim 1, wherein the threaded body further comprises a shaft comprising a tapered shape, wherein the shaft tapers from the threaded body second end toward the threaded body first end.

4. The compression device assembly of claim 1, wherein the nitinol compression element is cannulated for receiving a guidewire through the nitinol compression element.

5. The compression device assembly of claim 1, wherein the one or more pins of the insertion tool has a length configured such that a maximum stretch length of the nitinol compression element does not exceed a failure stretch length of the nitinol compression element.

6. The compression device assembly of claim 1, wherein the one or more pins of the insertion tool has a length of between about 1.0 mm to about 15.0 mm.

7. The compression device assembly of claim 1, wherein the setscrew is configured to engage the nitinol compression element to maintain the nitinol compression element in the deformed state prior to insertion into the patient.

8. The compression device assembly of claim 1, wherein the insertion tool is configured to receive a connection bolt through the insertion tool hollow interior.

9. The compression device assembly of claim 8, wherein the connection bolt further comprises a first end body configured to interface to a first end inner boundary of the insertion tool.

10. The compression device assembly of claim 8, wherein the connection bolt is configured to attach to one or more threads of the sliding element.

11. The compression device assembly of claim 1, wherein the setscrew comprises threads to attach to one or more threads of the sliding element.

12. A method comprising:
inserting a compression device assembly into a patient, wherein the compression device assembly comprises:
an elongate threaded body comprising:
a threaded body first end comprising one or more screw threads; and
a threaded body second end comprising an opening to a hollow interior of the threaded body;
a sliding element defining a sliding element hollow interior and comprising:
a sliding element first end comprising an elongated portion shaped to interface with the opening of the threaded body second end and comprising one or more pin slots configured for receiving one or more pins of an insertion tool;
a sliding element second end defining one or more pin openings configured for receiving the one or more pins of the insertion tool, wherein the insertion tool defines a hollow interior of the insertion tool and comprises one or more pins configured to be received by the one or more pin openings and the one or more pin slots of the sliding element;
a setscrew configured to maintain a position and orientation of a nitinol compression element within the compression device assembly, wherein the nitinol compression element is operatively connected to the threaded body, the setscrew, and the sliding element;
sliding the compression device assembly over a guidewire via a cannulation extending through the nitinol compression element, the threaded body, and the sliding element, such that the compression device assembly is guided to a target site within the patient;
contacting the threaded body with a first skeletal element of the patient via the one or more screw threads;
contacting the sliding element with a second skeletal element of the patient; and
disengaging the insertion tool from the sliding element and withdrawing the insertion tool from the patient; and
applying compression to the first skeletal element and the second skeletal element via the nitinol compression element returning to a relaxed state from a deformed state.

13. The method of claim 12, wherein the nitinol compression element further comprises a second end comprising a slotted end configured to receive an insert.

14. The method of claim 12, wherein the threaded body further comprises a shaft comprising a tapered shape, wherein the shaft tapers from the threaded body second end toward the threaded body first end.

15. The method of claim 12, wherein the insertion tool comprises three pins configured to be received by the one or more pin openings of the sliding element.

16. The method of claim 12, wherein the one or more pins of the insertion tool has a length configured such that a maximum stretch length of the nitinol compression element does not exceed a failure stretch length of the nitinol compression element.

17. The method of claim 12, wherein the one or more pins of the insertion tool has a length of between about 1.0 mm and about 15.0 mm.

18. The method of claim 12, wherein the setscrew is configured to maintain the nitinol compression element position within the sliding element.

19. The method of claim 12, wherein the insertion tool is configured to receive a connection bolt through the insertion tool hollow interior.

20. The method of claim 19, wherein the connection bolt further comprises a first end body configured to interface with a first end inner boundary of the insertion tool.

21. The method of claim 19, wherein the connection bolt is configured to attach to one or more threads of the sliding element.

22. The method of claim 21, wherein the setscrew comprises threads configured to interface with one or more threads of the sliding element.

23. A kit comprising:
a compression device assembly comprising:
(iii) a threaded body;
(iv) a sliding element;
(v) a setscrew; and
(v) a nitinol compression element;
a connection bolt;
one or more surgical devices; and
an insertion tool,
wherein:
the connection bolt, the nitinol compression element, and the setscrew are screwed into threads of the sliding element;
the nitinol element passes through the threaded body; and
the connection bolt passes through the insertion tool.
